(19) United States
(12) Patent Application Publication
(10) Pub. No.: US 2004/0138636 A1
(43) Pub. Date: Jul. 15, 2004

(54) Sanitary napkin or panty liner with staggered foldable wings

(76) Inventors: Jeannine Cardin, St-Eugene (CA); Eve-Marie Roy, Drummondville (CA)

Correspondence Address: ST. ONGE STEWARD JOHNSTON & REENS, LLC, 986 BEDFORD STREET, STAMFORD, CT, 06905-5619, US

(21) Appl. No.: 10/744,568

(22) Filed: Dec. 23, 2003

(30) Foreign Application Priority Data
Dec. 23, 2002 (CA) 2,415,060

(51) Int. Cl. A61F 13/15; A61F 13/20

(52) U.S. Cl. 604/385.04

(57) ABSTRACT

The invention relates to a sanitary napkin and/or panty liner with staggered foldable adhesive wings that do not overlap nor bunch when folded under the crotch of a regular or a narrow crotch type of undergarment.

### FIELD OF THE INVENTION

The present invention relates generally to the filed of sanitary napkins and/or panty liners having foldable wings that attach the napkin to the underside portion of an undergarment. More particularly, it concerns a sanitary napkin or panty liner which has foldable wings in a staggered relationship with each other thereby preventing the wings from overlapping when they are folded and fastened to the undergarment.

### BACKGROUND OF THE INVENTION

Already known in the prior art, are the sanitary pads or panty liners possessing a longitudinal main part with lateral foldable flaps or wings attached thereon. These flaps or wings are meant to fold under and adhere to the panty so as to hold the pad or liner securely in place. Depending on the crotch width and the design of the flaps or wings, the flaps or wings could possibly adhere to each other and to the undergarment of the wearer, as shown in FIGS. 5 and 6 which are views of prior art napkins.

U.S. Pat. No. 5,401,268 describe a sanitary napkin with adhesive wings that are asymmetrical and of triangular form. The lateral wings disclosed therein are both on the same transversal axis which is perpendicular relative to axis of the main longitudinal part of the pad or napkin. Furthermore, this prior art document also discloses a head-to-tail arrangement of the foldable wings.

Various other documents of prior art such as U.S. Pat. No. 6,312,418 B1, U.S. Pat. No. 6,387,084 B1, U.S. Pat. No. 5,772,650, D348,514 and U.S. Pat. No. 4,936,839 show sanitary napkins which have lateral adhesive wings that are generally rectangular in shape.

These documents are typical of the prior art in that they disclose lateral wings that are positioned in a symmetrical manner on either side of the sanitary napkin. These wing arrangements however pose a series of drawbacks for the users.

One of these drawbacks is due to the fact that very often these symmetrical lateral wings fold under the undergarment and overlap onto each other. Overlapping of the adhesive part of the wings underneath the undergarment entails a series of inconveniences for the user. The main drawback or inconvenience is due to the fact that overlapping adhesive wings require a fair degree of dexterity on the part of the user in order to both put the sanitary napkin correctly into place and to remove it. The delicate removal of a soiled napkin or pad is therefore only compounded because the more difficult the removal the more likely the user is to use excessive force and inadvertently end up tearing the wings off of the sanitary napkin and/or damaging the undergarment. Another drawback or inconvenience is the tendency of the folded wings to “bunch up” underneath the undergarment and cause discomfort to the user in a highly sensitive area of the anatomy.

Moreover, because of the fact that the relevant part of the wearer's undergarment is neither perfectly flat nor perfectly rectangular, other prior art wings still have a tendency in actual fact to overlap even when they are not intended or purported not to do so, such as in U.S. Pat. No. 5,401,268.

Another of the important drawbacks of these prior art sanitary napkin wings, is due to the fact that they are not adapted to certain styles of undergarment that are presently gaining in popularity. These undergarments are known as “thong” or “g-string” type undergarments. These undergarments have a narrow crotch. Due to the narrowness of the crotch of those types of undergarments, the chance for the wings to overlap, the importance of the overlapping and the bunching up problem are increasing, as well as the difficulty to take off the pad from the undergarment.

Yet another important drawback associated with these types of lateral wing napkins is their production. Certain products on the market have Velcro wings or wings which eliminate some of the problems stated above. However, the cost of production of such napkins with Velcro wings is relatively expensive. Another problem associated with such production is its relative complexity which entails that it is not easily adaptable to most types of manufacturing equipment.

In view of the above, the prior art does not provide a common solution to the separate problems of handling, bunching, adaptability to “thong” and “g-string” style undergarments, and production.

Thus, there exists a definite need for sanitary napkins which have wings that fold underneath the undergarment but do not overlap and which are adaptable to different types of napkins as well as to different types of undergarments, notably “g-string” or “thong” type undergarments, and that are easily and cost effectively produced.

The sanitary napkin of the present invention is distinct from and overcomes several disadvantages of the prior art, as will be discussed in detail below.

### SUMMARY OF THE INVENTION

An object of the present invention is to provide a sanitary napkin that overcomes several of the above mentioned drawbacks.

In accordance with the present invention, that object is achieved with a sanitary napkin suitable for use in a crotch portion of an undergarment, comprising an elongated absorbent main body extending on a longitudinal axis for laying on an upper side of the crotch portion; the main body having longitudinally opposed front and rear ends and transversally opposed side edges. The napkin further comprises at least one pair of lateral foldable wings, with adhesive fastener thereon, projecting outwardly from the opposed side edges to be folded and fastened to the underside of the crotch portion. The wings are characterized in that they are in a staggered relationship with each other. They are thus advantageously prevented from overlapping when they are folded and fastened to the underside of the crotch portion.

Another advantage of a sanitary napkin according to the present invention is the prevention of the “bunching up” problem, making the sanitary napkin more comfortable to the wearer and easier to remove.

It is worth mentioning that by sanitary napkin, it is meant any absorbent pad, panty shield, napkin or liner of any thicknesses, lengths or absorbance capacities used by women during their periods.

Another advantage found with a sanitary napkin of the invention is its adaptability to different types of undergarments, especially those with a narrow crotch, such as G-string or thong string. Indeed, in a first preferred embodiment of the present invention, the sanitary napkin has a main body with a general triangular shape, with the front end being larger than the rear end to fit the crotch portion of a G-string undergarment or any other undergarment with a narrow crotch. In that case, the front end of the napkin may preferably be slightly concave whereas the rear end is convex. More preferably, the radius of the concave front end is equal to the radius of the convex rear end, in order to limit the amount of scraping during the cutting procedure of the manufacture process.

However, in a second preferred embodiment, the main body is generally rectangular in shape with the side edges being longer than the front and rear ends.

In accordance with a further preferred aspect of the invention, the sanitary napkin comprises lateral bands from which the lateral foldable wings project. The lateral bands are contiguous the side edges of the main body and have variable length and width depending on the type of sanitary napkin produced. Preferably, the lateral bands are shorter than the side edges and are situated in their middle portion. Such lateral bands offer an added protection to the wearer.

In accordance with a still preferred aspect of the invention, the wings are aligned along transversal axes that run parallel to each other and perpendicular to the longitudinal axis of the elongated absorbent main body. However, in another preferred embodiment, the lateral foldable adhesive wings are aligned along axes that intersect each other and are at an angle higher or lower than 90° to the longitudinal axis of the elongated absorbent main body.

The foldable wings may take various shapes, as for example, they can have a rectangular, triangular or irregular shape.

The present invention is also directed to a sanitary napkin suitable for use in a crotch portion of a G-string undergarment or any other undergarment with a narrow crotch. The sanitary napkin comprises an elongated absorbent generally triangular shaped main body extending on a longitudinal axis for laying on an upper side of a crotch portion of the undergarment. The main body has longitudinally opposed front and rear ends where the front end is larger than the rear end and transversally opposed side edges. The napkin further comprises at least one pair of lateral foldable wings with adhesive fastener thereon. The wings are projecting outwardly from the opposed side edges to be folded and fastened to the underside of the crotch portion of an undergarment. The wings are also in a staggered relationship with each other thereby preventing overlapping when folded and fastened to the underside of the crotch portion of an undergarment.

### BRIEF DESCRIPTION OF THE DRAWINGS

These and other objects and advantages of the invention will become apparent upon reading the detailed description and upon referring to the drawings in which:

FIG. 1 is a top view of a sanitary napkin according to a first preferred embodiment of the invention suitable for use with a thong or g-string type of undergarment, showing the lateral staggered wings unfolded.

FIG. 2 is a bottom view of a sanitary napkin according to a variant of the preferred embodiment shown in FIG. 1, positioned and attached to a thong or g-string type of undergarment.

FIG. 3 is a top view of a sanitary napkin according to a second preferred embodiment, showing the lateral wings unfolded.

FIG. 4 is a bottom view of a sanitary napkin according to a variant of the embodiment shown in FIG. 3, positioned and attached to a conventional large crotch undergarment.

FIG. 5 is a bottom view of a prior art napkin or pad in a thong or g-string type of undergarment showing the lateral wings attached to the undergarment.

FIG. 6 is a bottom view of a prior art napkin or pad suitable for use with a conventional large crotch undergarment, attached to the undergarment.

FIG. 7 is a top view of a sanitary napkin according to another preferred embodiment of the invention suitable for use with a thong or g-string type of undergarment, showing the lateral staggered wings unfolded aligned along non parallel intersecting axes

While the invention will be described in conjunction with example embodiments, it will be understood that it is not intended to limit the scope of the invention to such embodiments. On the contrary, it is intended to cover all alternatives, modifications.

### DESCRIPTION OF PREFERRED EMBODIMENTS

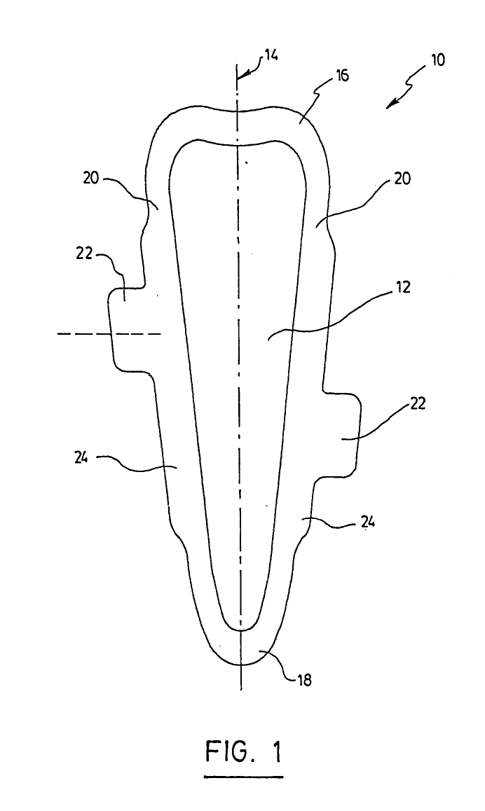

Referring to either one of FIGS. 1 to 4 and 7, a sanitary napkin (10) according to the invention comprises an elongated absorbent main body (12) extending on a longitudinal axis (14), having longitudinally opposed front and rear ends (16, 18); and transversally opposed side edges (20). In use, and as for conventional sanitary napkin, the main absorbent body (12) is laying on an upper side of the crotch portion (1) of an undergarment (2).

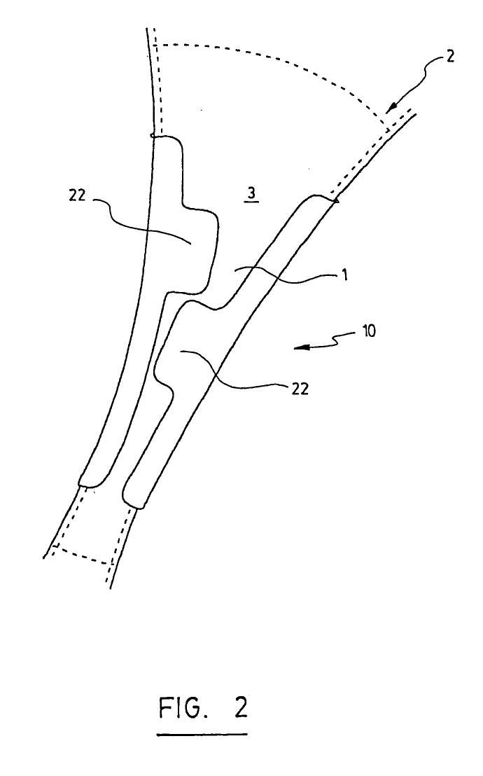

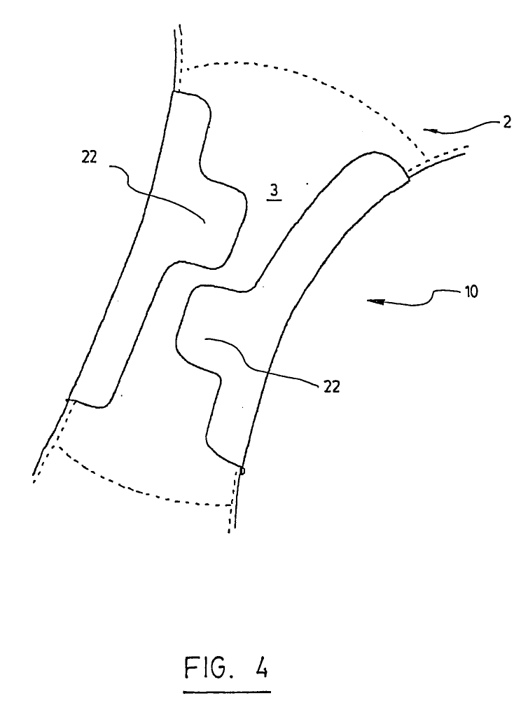

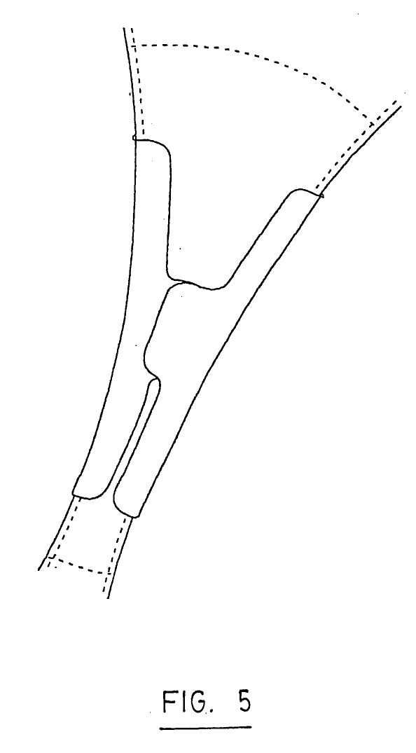

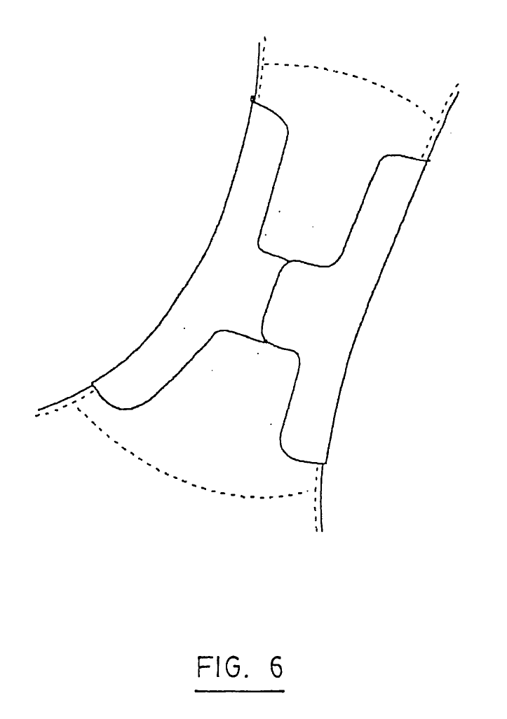

The napkin (10) further comprises a pair of lateral foldable wings (22) with adhesive fastener thereon (not illustrated). The wings (22) are projecting outwardly from the opposed side edges (20) to be folded and fastened to the underside (3) of the crotch portion (1) of the undergarment. As can be appreciated, the wings (22) are in a staggered relationship with each other. Hence, they are prevented from overlapping when folded and fastened to the underside (3) of the crotch portion (1), as best shown in FIGS. 2 and 4. As can be appreciated, the two wings (22) in an unfolded position, as in FIGS. 1, 3 or 7, are aligned along distinct transversal axes. In other words, these two wings (22) are not facing each other. Therefore, when the two wings (22) are in folded position, they adhere to two distinct and separate regions of the crotch region (1) and not to each other, as in the napkins of the prior art shown in FIGS. 5 and 6. This has the advantage of avoiding overlapping of the folded wings and hence avoids uncomfortable and inconvenient bunching of the folded wings underneath the undergarment. This in turn makes for easier and more convenient installation and removal for the user.

Turning now to FIGS. 1, 2 and 7, a sanitary napkin (10) suitable for use with G-string, thong string or any other narrow crotch undergarment is illustrated. In that particular case, the main body (12) has a generally triangular shaped that fits the shape of a thong string or g-string. More specifically, in that case the front end (16) is larger than the rear end (18). As best seen in FIG. 1, the front end (16) is preferably concave and has the same radius as the rear convex end (18). This allows the reduction of scrapping of material during manufacture.

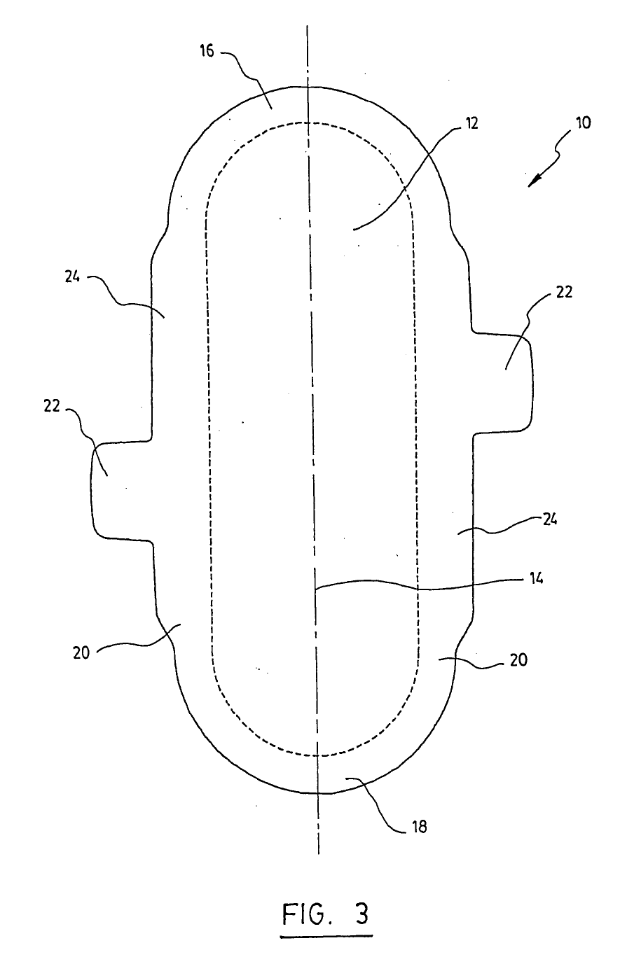

Referring now to FIGS. 3 and 4, the sanitary napkin (10) according to a second preferred embodiment, the main body (12) has a more conventional generally rectangular shape with the side edges (20) being longer than the front and rear ends (16, 18). In that case, the front and rear ends (16, 18) are preferably convexe.

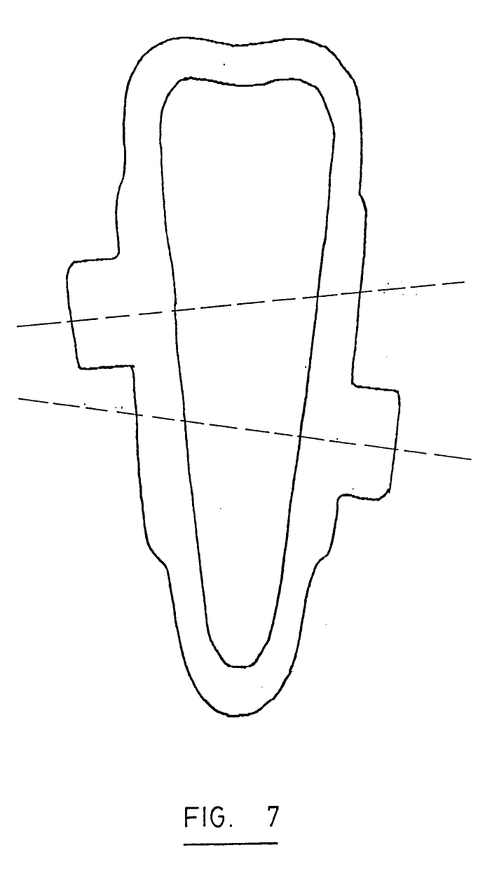

In both preferred embodiments shown in FIGS. 1 to 4, the wings (22) are preferably aligned along transversal axes that run parallel to each other and which run perpendicular to the longitudinal axis (14) of the main body (12). It is however worth mentioning that in another embodiment of the invention as shown in FIG. 7, the wings may very well be aligned along non parallel intersecting axes.

As it can be appreciated by referring to FIGS. 1 and 3, in a preferred variant of the invention, the sanitary napkin (10) comprises lateral bands (24) from which the lateral foldable wings (22) project. The lateral bands (24) are contiguous to each one of the side edges (20) of the main body (12) and have variable length and width depending on the type of the sanitary napkin produced. Preferably, the lateral bands (24) are shorter than the side edges (20) and are situated in their middle portion. More preferably, the lateral bands (24) are 12 mm in width. Such lateral bands (24) offer an added protection to the wearer.

A sanitary napkin according to the invention is preferably composed of three parts (not shown). From the top to the bottom: 1) top sheet, 2) an absorbent core and 3) a back sheet. Each one of these layers will now be described in more details.

1) The top sheet is preferably made of a hydrophilic material to let the liquid enter the main absorbent core as fast as possible. More preferably, it is made of a material selected from the group consisting of an apertured film coversheet, a nonwoven coversheet, an apertured nonwoven coversheet, a combination thereof and a lamination of a combination thereof. It can also be made of any other equivalent material or combination of materials.

2) The absorbent core can comprise any material usually described in the prior art. More preferably it is made of a material selected from the group consisting of comminuted wood pulp, creped cellulose tissue, airlaid, absorbent sponges, polymeric fibers, synthetic staple fibers, and a combination thereof. The airlaid comprises super absorbent material in powder or in fiber form; and it is thermally bonded, latex bonded or a combination thereof.

3) The back sheet is preferably waterproof and made of a material selected from the group consisting of a polyethylene thermoplastic film, a polypropylene thermoplastic film, a polyolefin, a foam, a woven material, a nonwoven material and a combination thereof. The back sheet serves as a barrier to the liquid to stop the liquid absorption just before touching the undergarment of the wearer.

All those layers are held together by different application of adhesives.

The lateral foldable wings (22) may also comprise a top sheet and a back sheet which are respectively an integral part of the top sheet and back sheet of the main body. It is however worth mentioning that the wings could also comprise only a back sheet which is an integral part of the back sheet of the main body.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

What is claimed is:

1. A sanitary napkin suitable for use in a crotch portion of an undergarment, the sanitary napkin comprising:
an elongated absorbent main body extending on a longitudinal axis for laying on an upper side of said crotch portion of the undergarment, said main body having longitudinally opposed front and rear ends; and transversally opposed side edges; and
at least one pair of lateral foldable wings with adhesive fastener thereon projecting outwardly from said opposed side edges to be folded and fastened to an underside of said crotch portion of the undergarment, said wings being in a staggered relationship with each other thereby preventing the wings from overlapping when folded and fastened to the underside of said crotch portion of the undergarment.

2. The sanitary napkin according to claim 1, wherein the main body has a general triangular shape with the front end being larger than the rear end to fit the crotch portion of a G-string undergarment or an undergarment with a narrow crotch.

3. The sanitary napkin according to claim 2, wherein the front end is slightly concave.

4. The sanitary napkin according to claim 1, comprising a lateral band contiguous to each one of said side edges, said lateral bands being shorter than said side edges and being situated in a middle portion of thereof.

5. The sanitary napkin according to claim 4, wherein said wings are projecting from said lateral bands.

6. The sanitary napkin according to claim 1, wherein the lateral foldable adhesive wings are aligned along transversal axes that run parallel to each other, said transversal axes run perpendicular to the longitudinal axis of the elongated absorbent main body.

7. The sanitary napkin according to claim 1, wherein the lateral foldable adhesive wings are aligned along transversal axes that run at an angle higher or lower than 90° relative to each other.

8. The sanitary napkin according to claim 1, wherein the main body has a rectangular shape with the side edges being longer than the front and rear ends.

9. The sanitary napkin according to 1, wherein the foldable wings are rectangular in shape.

10. The sanitary napkin according to claim 1, wherein the elongated body comprises a top sheet, an absorbent core and a back sheet.

11. The sanitary napkin according to claim 10, wherein the top sheet is made of a hydrophilic material to let the liquid enter the main absorbent core as fast as possible.

12. The sanitary napkin according to claim 11, wherein said top sheet is made of a material selected from the group consisting of an aperture film coversheet, a nonwoven coversheet, an apertured nonwoven coversheet, a combination thereof and a lamination of a combination thereof.

13. The sanitary napkin of claim 10, wherein the absorbent core is made of a material selected from the group consisting of comminuted wood pulp, creped cellulose tissue, airlaid, absorbent sponges, polymeric fibers, synthetic staple fibers, and a combination thereof.

14. The sanitary napkin of claim 13, wherein the airlaid comprises superabsorbent material in powder or in fiber form.

15. The sanitary napkin of claim 13, wherein the airlaid is thermally bonded, latex bonded or a combination thereof.

16. The sanitary napkin of claim 10, wherein the backsheet is waterproof.

17. The sanitary napkin of claim 16, wherein the backsheet is made of a material selected from the group consisting of a polyethylene thermoplastic film, a polypropylene thermoplastic film, a polyolefin, a foam, a woven material, a nonwoven material and a combination thereof.

18. The sanitary napkin according to claim 10, wherein the top sheet, the absorbent core and the back sheet are bonded to each other.

19. The sanitary napkin according to claim 10, wherein the lateral foldable wings comprise a top sheet and a back sheet which are respectively an integral part of the top sheet and back sheet of the main body.

20. A sanitary napkin suitable for use in a crotch portion of a G-string undergarment or an undergarment with a narrow crotch, the sanitary napkin comprising:
an elongated absorbent generally triangular shaped main body extending on a longitudinal axis for laying on an upper side of said crotch portion of the undergarment, said main body having longitudinally opposed front and rear ends; and transversally opposed side edges; the front end being larger than said rear end; and
at least one pair of lateral foldable wings with adhesive fastener thereon projecting outwardly from said opposed side edges to be folded and fastened to an underside of said crotch portion of the undergarment, said wings being in a staggered relationship with each other thereby preventing the wings from overlapping when folded and fastened to the underside of said crotch portion of the undergarment.
